(19) United States
(12) Patent Application Publication
(10) Pub. No.: US 2005/0032031 A1
(43) Pub. Date: Feb. 10, 2005

(54) Method for eliminating fragile cells from stored cells

(76) Inventors: John H. Crowe, Davis, CA (US); Fern Tablin, Davis, CA (US); Nelly M. Tsvetkova, Davis, CA (US); Zsolt Torok, Davis, CA (US); Gyana R. Satpathy, Davis, CA (US); Denis M. Dwyre, Iowa City, IA (US); Rachna Bali, West Sacramento, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/635,754

(22) Filed: Aug. 6, 2003

(51) Int. Cl. A01N 1/02; C12N 5/08

(52) U.S. Cl. 435/2; 435/372

(57) ABSTRACT

A method for reducing hemolysis in cells including washing cells in a solute solution having the capabilities of reducing cell hemolysis by at least about 0.50% for each 100 mOsm increase in osmolarity of the solute solution. A cell produced by the method for reducing hemolysis. The method permits removal of osmotically fragile cells from the population.

### RELATED PATENT APPLICATIONS

This patent application is related to co-pending patent application Ser. No. 10/052,162, filed Jan. 16, 2002. Patent application Ser. No. 10/052,162 is a continuation-in-part patent application of co-pending patent application Ser. No. 09/927,760, filed Aug. 9, 2001. Patent application Ser. No. 09/927,760 is a continuation-in-part patent application of co-pending patent application Ser. No. 09/828,627, filed Apr. 5, 2001. Patent application Ser. No. 09/828,627 is a continuation patent application of patent application Ser. No. 09/501,773, filed Feb. 10, 2000. All of the foregoing patent applications are fully incorporated herein by reference thereto as if repeated verbatim immediately hereinafter.

### STATEMENT REGARDING FEDERAL SPONSORED RESEARCH AND DEVELOPMENT

Embodiments of this invention were made with Government support under Grant No. N66001-00-C-8048, awarded by the Department of Defense Advanced Research Projects Agency (DARPA). Further embodiments of this invention were made with Government support under Grant Nos. HL57810 and HL61204, awarded by the National Institutes of Health. The Government has certain rights to embodiments of this invention.

### FIELD OF THE INVENTION

Embodiments of the present invention generally broadly relate to living mammalian cells. More specifically, embodiments of the present invention generally provide for the preservation and survival of cells, especially human cells, such as erythrocytic cells, and for reducing hemolysis and eliminating osmotically-fragile cells.

The compositions and methods for embodiments of the present invention are useful in many applications, such as in medicine, pharmaceuticals, biotechnology, and agriculture, and including transfusion therapy, as hemostasis aids and for drug delivery.

### BACKGROUND OF THE INVENTION

A cell is broadly regarded in the art as a small, typically microscopic, mass of protoplasm bounded externally by a semi-permeable membrane, usually including one or more nuclei and various other organelles with their products. A cell is capable either alone or interacting with other cells of performing all the fundamental function(s) of life, and forming the smallest structural unit of living matter capable of functioning independently.

Cells may be transported and transplanted; however, this requires cryopreservation which includes freezing and subsequent reconstitution (e.g., thawing, re-hydration, etc.) after transportation. Unfortunately, a very low percentage of cells retain their functionality after undergoing freezing and thawing. While some cryoprotectants, such as dimethyl sulfoxide, tend to lessen the damage to cells, they still do not prevent some loss of cell functionality.

Trehalose has been found to be suitable in the cryopreservation of cells and platelets. Trehalose is a disaccharide found at high concentrations in a wide variety of organisms that are capable of surviving almost complete dehydration. Trehalose has been shown to stabilize membranes, proteins, and certain cells during freezing and drying in vitro.

U.S. Pat. No. 5,827,741, Beattie et al., issued Oct. 27, 1998, discloses cryoprotectants for human cells and platelets, such as dimethylsulfoxide and trehalose. The cells or platelets may be suspended, for example, in a solution containing a cryoprotectant at a temperature of about 22° C. and then cooled to below 15° C. This incorporates some cryoprotectant into the cells or platelets, but not enough to prevent hemolysis of a large percentage of the cells or platlets.

Accordingly, a need exists for the effective and efficient preservation of cells. More specifically, and accordingly further, a need also exists for the effective and efficient cryopreservation of cells (e.g., erythrocytic cells, eukaryotic cells, or any other cells, and the like), such that the preserved cells respectively maintain their biological properties and may readily become viable after storage while hemolysis of the cells is reduced.

### SUMMARY OF EMBODIMENTS OF THE INVENTION

In one aspect of the present invention, a dehydrated composition is provided having a generally dehydrated composition comprising freeze-dried cells selected from a mammalian species (e.g., a human) and being effectively loaded internally (e.g., producing hyper-osmotic pressure on the cells to uptake external trehalose via fluid phase endocytosis) with at least about 10 mM of a carbohydrate (e.g., an oligosaccharide, such as trehalose) therein to preserve biological properties during freeze-drying and re-hydration. The amount of the carbohydrate inside the freeze-dried cells is preferably the amount obtained from maintaining a positive loading gradient or loading efficiency gradient on the cell. When the carbohydrate is trehalose, the amount of trehalose loaded inside the freeze-dried cells is preferably from about 10 mM to about 50 mM.

In another aspect of the present invention, a method is provided for loading (e.g., by fluid phase endocytosis) a solute into a cell (e.g., an erythrocytic cell). Embodiments of the invention include disposing a cell in a solution having a solute concentration of sufficient magnitude to produce hyper-osmotic pressure on the cell for transferring a solute (e.g., an oligosaccharide, such as trehalose) from the solution into the cell. The method may additionally comprise preventing a decrease in a loading efficiency gradient in the loading of the solute into the cell. In an embodiment of the invention where the solute comprises an oligosaccharide, the preventing a decrease in a loading efficiency gradient in the loading of the oligosaccharide into the cell may comprise maintaining a concentration of the oligosaccharide in the oligosaccharide solution below a certain concentration, such as below from about 35 mM to about 65 mM, more particularly below a concentration ranging from about 40 mM to about 60 mM, more particularly further below a concentration ranging from about 45 mM to about 55 mM (e.g., below about 50 mM). In another embodiment of the invention, the preventing a decrease in a loading efficiency gradient in the loading of the oligosaccharide into the cell comprises maintaining a positive gradient of loading efficiency to concentration of the oligosaccharide in the oligosaccharide solution.

The solute concentration includes an extracellular cellular solute concentration for elevating extracellular osmolarity within the solution to a value which is greater than a value of the intracellular osmolarity of the cell. The transferring of the solute is preferably by fluid phase endocytosis and preferably without degradation of the solute. In embodiments of the invention where the cell is an erythrocytic cell and the solute comprises trehalose, a gradient of trehalose (M) within the erythrocytic cell to extracellular trehalose concentration (M) within the solution may range from about 0.130 to about 0.200, particularly for a temperature ranging from about 300 C to about 40° C. (e.g., about 370 C). In a further embodiment of the invention, a gradient of trehalose (M) within the erythrocytic cell to extracellular trehalose concentration (M) within the solution ranges from about 0.04 to about 0.12, particularly for a temperature ranging from about 0° C. to about 10° C. In yet a further embodiment, a gradient of trehalose (M) within the erythrocytic cell to extracellular trehalose concentration (M) within the solution may range from about 0.04 to about 0.08, or from about 0.08 to bout 0.12, particularly for a temperature ranging from about 00 C to about 10° C. The solute solution may have a trehalose concentration ranging from about 320 mM to about 4000 mM, such as including from about 320 mM to about 2000 mM or from about 500 mM to about 1000 mM.

A further embodiment of the invention provides retaining the solute in the cell; more specifically, washing the cell and retaining the solute in the cell during the washing. The washing is with a washing buffer, and retention of the solute in the cell increases from about 25% to about 175% when a buffer concentration (e.g., the osmolarity of all osmotically active particles within the washing buffer solution) increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). The washing of the cell with a washing buffer includes employing a ratio of an extracellular buffer concentration (mOsm) to an intracellular solute concentration (mM) ranging from about 14.0 to about 4.0, such as from about 12.0 to about 5.0, including from about 9.0 to about 6.0 and from about 8.0 to about 7.0 (e.g., about 7.5).

Additional embodiments of the present invention provide a method for loading trehalose into an erythrocytic cell. The method may comprise disposing an erythrocytic cell in a trehalose solution having a trehalose concentration of at least about 25% (preferably at least about 50%) greater than the intracellular osmolarity of the erythrocytic cell for loading (e.g., by fluid phase endocytosis) the trehalose into the erythrocytic cell.

The loading of the trehalose from the trehalose solution into the erythrocytic cell may be without degradation of the trehalose, and produces a loaded erythrocytic cell having a gradient of loaded trehalose (M) within the erythrocytic cell to extracellular trehalose concentration (M) within the trehalose solution ranging from about 0.130 to about 0.200. In another embodiment, the loading of the trehalose produces a loaded erythrocytic cell having a gradient of loaded trehalose (M) within the erythrocytic cell to extracellular trehalose concentration (M) within the trehalose solution ranging from about 0.04 to about 0.12. In a further embodiment, the loading of the trehalose produces a loaded erythrocytic cell having a gradient of loaded trehalose (M) within the erythrocytic cell to extracellular trehalose concentration (M) within the trehalose solution ranging from about 0.04 to about 0.08, or from about 0.08 to about 0.12, depending on the extracellular trehalose concentration and the temperature of the trehalose solution. The trehalose solution may have a trehalose concentration ranging from about 25% to at least about 1000% greater than the intracellular osmolarity of the erythrocytic cell, or at least about 50% greater than the intracellular osmolarity of the erythrocytic cell.

A further embodiment of the invention provides retaining the trehalose in the erythrocytic cell; more specifically washing the erythrocytic cell and retaining the trehalose in the erythrocytic cell during the washing.

The washing of the erythrocytic cell is preferably with a washing buffer, and retention of the trehalose in the erythrocytic cell increases from about 25% to about 175% when a buffer concentration increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). The washing of the erythrocytic cell with a washing buffer includes employing a ratio of an extracellular buffer concentration (mOsm) to an intracellular trehalose concentration (mM) ranging from about 14.0 to about 4.0, more particularly from about 12.0 to about 5.0, including from about 9.0 to about 6.0 and from about 8.0 to about 7.0 (e.g., about 7.5).

Additional embodiments of the present invention provide a method for loading (e.g., by fluid phase endocytosis) an oligosaccharide into cells (e.g., erythrocytic cells) comprising disposing cells in an oligosaccharide solution having an oligosaccharide concentration of at least about 25% greater than the intracellular osmolarity of the cells for loading oligosaccharide into the cells, and preventing a decrease in a loading gradient in the loading of the oligosaccharide into the cells. In one embodiment of the invention, the preventing a decrease in a loading gradient in the loading of the oligosaccharide into the cells comprises maintaining a concentration of the oligosaccharide in the oligosaccharide solution below a certain concentration, such as below a concentration ranging from about 35 mM to about 65 mM, more particularly below a concentration ranging from about 40 mM to about 60 mM, more particularly further below a concentration ranging from about 45 mM to about 55 mM (e.g., below about 50 mM). In another embodiment the preventing a decrease in a loading gradient in the loading of the oligosaccharide into the cells comprises maintaining a positive gradient of concentration of oligosaccharide loaded into the cells to concentration of the oligosaccharide in the oligosaccharide solution.

Further embodiments of the present invention provide for a method for reducing hemolysis in cells. The method comprises washing cells in a solute solution having the capabilities of reducing cell hemolysis by at least about_{—}0.50% for each 100 mOsm increase in osmolarity of the solute solution. More specifically, the solute solution reduces cell hemolysis from about 0.50% to about 8.0% for each 100 mOsm increase in osmolarity of the solute solution, preferably reducing cell hemolysis from about 1.0% to about 4.0% for each 100 mOsm increase in osmolarity of the solute solution, more preferably reducing cell hemolysis from about 1.0% to about 2.0% for each 100 mOsm increase in osmolarity of the solute solution. The solute solution may comprise an osmolarity ranging from about 100 mOsm to about 1500 mOsm, preferably an osmolarity ranging from about 200 mOsm to about 1000 mOsm, more preferably an osmolarity ranging from about 300 mOsm to about 600 mOsm. The solute solution may comprise a salt solution having a phosphate buffered saline (PBS) solution including NaCl, Na₂HPO₄, and KH₂PO₄. More specifically, the solute solution comprises a PBS buffer having 154 mM NaCl, 5.6 mM Na₂HPO₄, 1.06 KH₂PO₄, and a pH of 7.2. The damaged cells may be removed from the washed cells, such as by centrifuging the washed cells, and the remaining cells after centrifuging and removing damaged cells may be suspended in the solute solution to facilitate storage of more robust cells.

A still further embodiment of the present invention provides a method for removing fragile cells from cells comprising washing cells in a solute solution having the capabilities of reducing cell hemolysis to produce washed cells including fragile cells; and removing the fragile cells from the washed cells. The solute solution has the capabilities of reducing hemolysis by at least about 0.50% for each 100 mOsm increase in osmolarity of the solute solution.

These provisions, together with the various ancillary provisions and features which will become apparent to those skilled in the art as the following description proceeds, are attained by the processes and cells of the present invention, preferred embodiments thereof being shown with reference to the accompanying drawings, by way of example only, wherein:

### BRIEF DESCRIPTION OF THE DRAWINGS

In the drawings:

FIG. 1 graphically illustrates the loading efficiency of trehalose plotted versus incubation temperature of human platelets;

FIG. 2 graphically illustrates the loading efficiency (cytosolic concentration divided by the extracellular concentration, the sum multiplied by 100) following incubation as a function of incubation time;

FIG. 3 graphically illustrates the internal trehalose concentration of human platelets versus external trehalose concentration as a function of temperature at a constant incubation or loading time;

FIG. 4 graphically illustrates the loading efficiency of trehalose into human platelets as a function of external trehalose concentration;

FIG. 5 graphically illustrates intracellular trehalose concentration in erythrocytic cells as a function of extracellular trehalose at respective temperatures of 4° C. and 37° C.;

FIG. 6 graphically illustrates the fragility index of erythrocytic cells incubated overnight at respective temperatures of 4° C. and 37° C. in the presence of and as a function of increasing intracellular trehalose concentrations;

FIG. 7 graphically illustrates trehalose uptake (i.e., intracellular trehalose mM) and hemolysis (i.e., % hemolysis) as a function of incubation temperature (0° C.);

FIG. 8 graphically illustrates intracellular trehalose concentration (mM) as a function of the osmolarity of the washing buffer;

FIG. 9 is a forward scatter vs. a side scatter flow cytometry for non-loaded (control) human erythrocytic cells in 300 mOsm PBS;

FIG. 10 is a forward scatter vs. aside scatter flow cytometry for trehalose-loaded human erythrocytic cells resuspended for 30 seconds in 300 mOsm PBS having a trehalose concentration of 60 mM;

FIG. 11 is a forward scatter vs. a side scatter flow cytometry for trehalose-loaded human erythrocytic cells resuspended for 5 minutes in 300 mOsm PBS having a trehalose concentration of 60 mM; and

FIG. 12 is a graphical illustration of hemolysis (%) vs. osmolarity of the PBS washing and incubation buffer for various washing incubation periods (min.)

### DETAILED DESCRIPTION OF PREFERRED EMBODIMENTS OF THE INVENTION

Compositions and embodiments of the invention include methods for loading solutes into cells, as well as cells that have been manipulated (e.g., by freeze-drying) or modified (e.g., loaded with a chemical or drug) in accordance with methods of the present invention. The cells may be any type of cell including, not by way of limitation, erythrocytic cells, eukaryotic cells or any other cell, whether nucleated or non-nucleated.

The term “erythrocytic cell” is used to mean any red blood cell. Mammalian, particularly human, erythrocytes are preferred. Suitable mammalian species for providing erythrocytic cells include by way of example only, not only human, but also equine, canine, feline, or endangered species.

The term “eukaryotic cell” is used to mean any nucleated cell, i.e., a cell that possesses a nucleus surrounded by a nuclear membrane, as well as any cell that is derived by terminal differentiation from a nucleated cell, even though the derived cell is not nucleated. Examples of the latter are terminally differentiated human red blood cells. Mammalian, and particularly human, eukaryotes are preferred. Suitable mammalian species include by way of example only, not only human, but also equine, canine, feline, or endangered species.

Broadly, the preparation of solute-loaded cells in accordance with embodiments of the invention comprises the steps of loading one or more cells with a solute by placing one or more cells in a solution having a solute concentration of sufficient magnitude to produce hyperosmotic pressure on the cell for transferring the solute from the solution into the cell. For increasing the transfer or uptake of the solute from the solute solution, the solute solution temperature or incubation temperature has a temperature above about 25° C., more preferably above 30° C., such as from about 30° C. to about 40° C. In another embodiment of the invention, a solute solution (e.g., trehalose solution) has a solute (e.g., trehalose) concentration of at least about 25%, preferably at least about 50%, greater than the intracellular osmolarity of the cells for loading the solute into the cells. For various embodiments of the invention, a solute solution has a solute concentration ranging from about 25% to at least about 1000% greater than the intracellular osmolarity of the cell. For additional various embodiments of the invention, the solute solution has a solute concentration ranging from about 320 mM to bout 4000 mM, preferably from about 320 mM to about 2000 mM, more preferably from about 500 mM to about 1000 mM. The method may additionally comprise preventing a decrease in a loading gradient and/or a loading efficiency gradient in the loading of the solute into the cells. Preventing a decrease in a loading efficiency gradient in the loading of the solute into the cells comprises maintaining a positive gradient of loading efficiency (e.g., in %) to concentration (e.g., in mM) of the solute in the solute solution. Preventing a decrease in a loading gradient in the loading of the oligosaccharide into the cells comprises maintaining a concentration of the solute in the solute solution below a certain concentration (e.g., below a concentration ranging from about 35 mM to about 65 mM, more particularly below from about 40 mM to about 60 mM, or below from about 45 mM to about 55 mM, such as below about 50 mM); and/or maintaining a positive gradient of concentration of solute loaded into the cells to concentration of the solute in the solute solution.

The solute solution may be any suitable physiologically acceptable solution in an amount and under conditions effective to cause uptake or “introduction” of the solute from the solute solution into the cells. A physiologically acceptable solution is a suitable solute-loading buffer, such as any of the buffers stated in the previously mentioned related patent applications, all having been incorporated herein by reference thereto.

The solute is preferably a carbohydrate (e.g., an oligosaacharide) selected from the following groups of carbohydrates: a monosaccharide (e.g., bioses, trioses, tetroses, pentoses, hexoses, heptoses, etc), a disaccharide (e.g., lactose, maltose, sucrose, melibiose, trehalose, etc), a trisaccharide (e.g., raffinose, melezitose, etc), or tetrasaccharides (e.g., lupeose, stachyose, etc), and a polysaccharide (e.g., dextrins, starch groups, cellulose groups, etc). More preferably, the solute is a disaccharide, with trehalose being the preferred, particularly since it has been discovered that trehalose does not degrade or reduce in complexity upon being loaded. Thus, in the practice of various embodiments of the invention, trehalose is transferred from a solution into the cells without degradation of the trehalose.

An extracellular medium of about 280-320 mOsm is considered iso-osmotic for cells, particularly erythrocytic cells, with regard to the amount of permeable solutes in the cytoplasm. Any increase of the amount of solutes in the extracelluar medium creates an osmotic shock, ranging from a mild shock at about 350 mM trehalose to a strong shock at about 4200 mM trehalose, and a leakage of water which would reversibly reduce the cell volume. However, small molecular weight solutes, such as trehalose, in an extracellular medium in a concentration higher than about 320 mM, can pass through the membrane of a cell using a diffusion vector. It has been discovered that an extracellular concentration of trehalose higher than about 450 mM (or mOsm), which is about 50% greater than an intracellular milliosmolarity, will produce an osmotic shock that will result in trehalose uptake. Increasing the extracellular trehalose concentration leads to even higher osmotic shock and higher trehalose uptake.

Molarity, or millimolarity, mM, is the number of moles (or millimoles) of a solute per liter of solution and is a measure of the concentration. Osmolarity (Osm), or milliosmolarity (mOsm), is a count of the number of dissolved particles per liter of solution and is a measure of the osmotic pressure exerted by solutes. Biological membranes, such as cell membranes, can be semi-permeable because they allow water and some small molecules to pass, but block the passage of proteins or macromolecules. Since the osmolarity of a solution is equal to the molarity times the number of particles per molecule, 600 mM trehalose is equal to 600 mOsm trehalose because trehalose does not dissociate in water. However, with respect to compounds that dissociate in water, such as NaCl, 1 mM NaCl is equal to 2 mOsm NaCl because it has two particles. Similarly, 100 mM NaCl is equal to 200 mOsm NaCl. Thus, for a 300 mOsm PBS buffer (154 mM NaCl, 5.6 mM Na₂HPO₄, 1.05 mm KH₂PO₄, pH 7.2), 300 mOsm refers to all of the osmotically active particles in the PBS solution, with 200 mOsm of the 300 mOsm stemming from NaCl.

Other embodiments of the present invention provide for retaining a solute in a cell. Preferably, after the cells have been loaded with a solute, such as an oligosaccharide (e.g., trehalose), the cells are then washed. More preferably, during the washing of the cells the solute is retained in the cells. Washing leads to hemolysis of the fragile cells and removal of cellular fragments and free hemoglobin. The net result is that the remaining cells do indeed have an elevated trehalose content. The washing may be with a washing solution (e.g., such as a washing buffer having an oligosaccharide), and retention of the solute in the cell increases from about 25% to about 175% when a buffer concentration (e.g., the osmolarity of all osmotically active particles within the washing buffer solution) increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). The washing of the cell with a washing buffer includes employing a ratio of a buffer concentration (e.g., an extracellular buffer concentration) (mOsm) to an intracellular solute concentration (mM) ranging from about 14.0 to about 4.0, such as from about 12.0 to about 5.0, including from about 9.0 to about 6.0 and from about 8.0 to about 7.0 (e.g., about 7.5).

As indicated in patent application Ser. No. 10/052,162, which claims the benefit of patent application Ser. No. 09/501,773, filed Feb. 10, 2000, with respect to common subject matter, the amount of the preferred trehalose loaded inside the cells ranges from about 10 mM to about 50 mM, and is achieved by incubating the cells to preserve biological properties during freeze-drying with a trehalose solution, preferably a trehalose solution that has up to about 50 mM trehalose therein. Higher concentrations of trehalose during incubation are not preferred, particularly since an embodiment of the invention includes preventing a decrease in a loading gradient, or a loading efficiency gradient, in the loading of the solute into the cell. It has been discovered that preventing a decrease in a loading gradient, or a loading efficiency gradient, in the loading of a oligosaccharide (i.e., trehalose) into a cell comprises maintaining a concentration of the oligosaccharide in the oligosaccharide solution below a certain concentration (e.g., below a concentration ranging from about 35 mM to about 65 mM, more particularly below from about 40 mM to about 60 mM, or below from about 45 mM to about 55 mM, such as below about 50 mM). It has been further discovered that preventing a decrease in a loading gradient, or a loading efficiency gradient, in the loading of a oligosaccharide (i.e., trehalose) into a cell comprises maintaining a positive gradient of loading efficiency to concentration of the oligosaccharide in the oligosaccharide solution.

As further indicated in co-pending patent application Ser. No. 10/052,162, the effective loading of trehalose is also accomplished by means of using an elevated temperature of from greater than about 25° C. to less than about 40° C., more preferably from about 30° C. to less than about 40° C., most preferably about 37° C. This is due to the discovery of the second phase transition for cells.

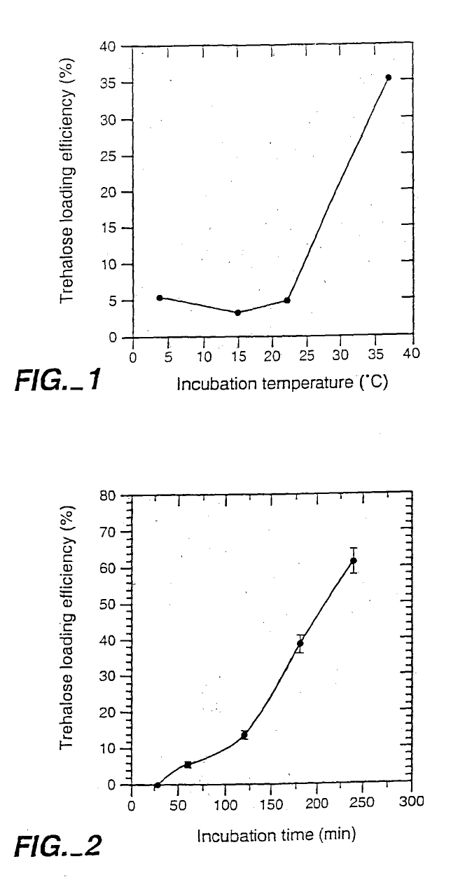

Referring now to FIG. 1, there is seen a graphical illustration from co-pending patent application Ser. No. 10/052,162 of the loading efficiency of trehalose plotted versus incubation temperature of human platelets. The trehalose loading efficiency begins a steep slope increase at incubation temperatures above about 25° C. and continues up to about 40° C. The trehalose concentration in the exterior solution (that is, the solute solution or loading buffer) and the temperature during incubation together lead to a trehalose uptake that occurs through fluid phase endocytosis. Example 1 below provides the more specific testing conditions and parameters which produced the graphical illustrations of FIG. 1. It is believed that the graphical illustration of the loading efficiency in FIG. 1 would be generally applicable for cells in general.

Referring now to FIG. 2, there is seen an illustration from co-pending patent application Ser. No. 10/052,162 of trehalose loading efficiency for human blood platelets as a function of incubation time. More specifically, FIG. 2 is a graphical illustration of the loading efficiency (cytosolic concentration divided by the extracellular concentration, the sum multiplied by 100) following incubation as a function of incubation time. Example 1 below provides the more specific testing conditions and parameters which produced the graphical illustrations of FIG. 2. It is believed that the graphical illustration of the loading efficiency in FIG. 2 would also be generally applicable for cells in general.

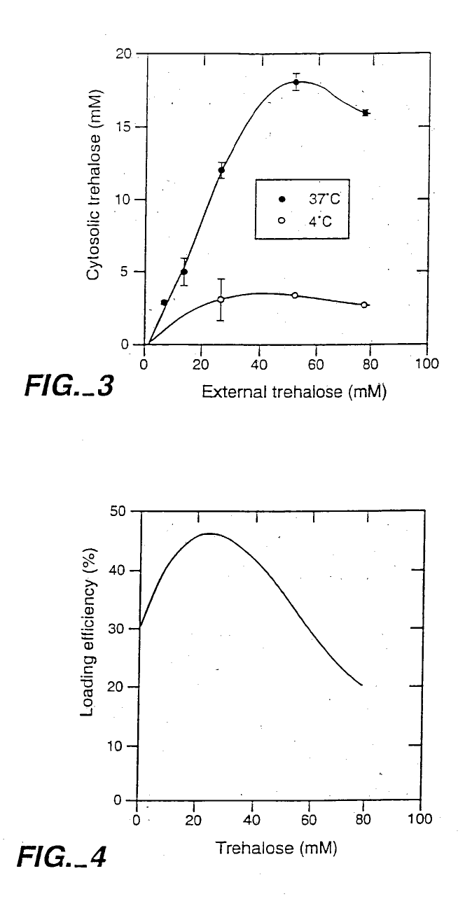

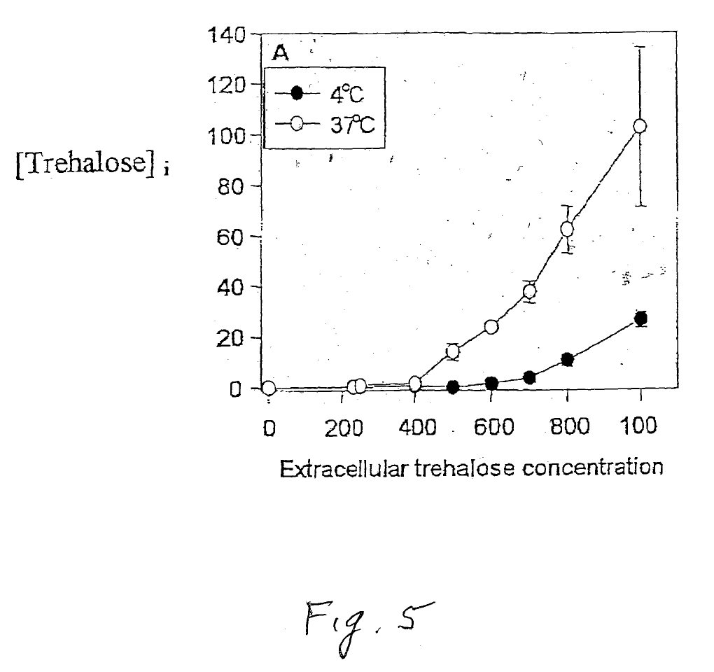

Referring now to FIG. 3, there is seen a graphical illustration from patent application Ser. No. 10/052,162 of the internal trehalose concentration of human platelets versus external trehalose concentration as a function of 4° C. and 37° C. temperatures at a constant incubation or loading time. In FIG. 4 there is seen a graphical illustration from patent application Ser. No. 10/052,162 of the loading efficiency of trehalose into human platelets as a function of external trehalose concentration. Example 1 below provides the more specific testing conditions and parameters which produced the graphical illustrations of FIGS. 3 and 4. In additional embodiments of the present invention, it is further believed that the general findings illustrated in FIGS. 3 and 4 with respect to platelets are generally broadly applicable to cells in general.

Thus, applying the findings illustrated in FIG. 3 and in FIG. 4 to solutes and cells in general, a decrease in a loading gradient or a loading efficiency gradient in the loading of a solute into a cell may be prevented. For an embodiment of the present invention and as broadly illustrated in FIG. 3, preventing a decrease in a loading gradient or a loading efficiency gradient in the loading of the solute (e.g., an oligosaccharide such as trehalose) into the cell comprises maintaining a concentration of the solute (e.g., an oligosaccharide such as trehalose) in the solute solution (e.g. an oligosaccharide solution such as a trehalose solution) below a solute concentration ranging from about 35 mM to about 65 mM, more specifically a solute concentration ranging from about 40 mM to about 60 mM, more specifically further a solute concentration ranging from about 45 mM to about 55 mM (e.g., about 50 mM). In another embodiment of the present invention and as best illustrated in FIG. 4, preventing a decrease in a loading gradient or a loading efficiency gradient in the loading of the solute (e.g., an oligosaccharide, such as trehalose) into the cell comprises maintaining a positive gradient of loading efficiency (e.g., loading efficiency in %) to concentration (e.g., concentration in mM) of the solute in the solute solution (e.g. an oligosaccharide solution, such as a trehalose solution).

When a solute is loaded from a solute solution into one or more cells, the solute solution preferably has a solute concentration of sufficient magnitude to produce hyperosmotic pressure on the one or more cells. It has been discovered that the basis for the loading of the solute into the cells is dependent upon osmotic shock. The magnitude of osmotic shock and hyperosmotic pressure on the cells depends on the difference between internal solute concentration, or the intracellular osmolarity, within the cells, and the external solute concentration within the solute solution, or the extracellular cellular solute concentration. For embodiments of the invention, the solute solution has a solute concentration ranging from about 320 mM to about 4000 mM, preferably from about 320 mM to about 2000 mM, more preferably from about 500 mM to about 1000 mM.

It has also been discovered that the basis for the loading of the solute into the cells is not only dependent upon osmotic shock, but is also dependent upon the thermal effects on flux of the solute across the membranes of the cells. The higher the thermal effects on flux of the solute across the membranes of the cells, the larger the amount of solute loaded into the cells. Stated alternatively, loading of a solute into cells increases as the temperature of the solute solution increases. Referring now to FIG. 5, there is seen a graphical illustration of intracellular trehalose concentration as a function of extracellular trehalose at respective temperatures of 4° C. and 37° C. Thus, at a temperature ranging from about 30° C. to about 40° C. (e.g. at about 37° C.) a gradient of a solute concentration (M), such as an oligosaccharide (e.g., trehalose) concentration, within a cell (e.g., an erythrocytic cell) to extracellular solute concentration (M) within a loading solution (or buffer) ranges from about 0.130 to about 0.200. At a temperature ranging from about 0° C. to about 10° C. (e.g. at about 4° C.) a gradient of a solute concentration (M), such as an oligosaccharide (e.g., trehalose) concentration, within a cell (e.g., an erythrocytic cell) to extracellular solute concentration (M) within a loading solution (or buffer) ranges from about 0.04 to about 0.12, more specifically from about 0.04 to about 0.08, and from about 0.08 to about 0.12, depending on the quantity of extracellular solute concentration. Example 2 below provides the more specific testing conditions and parameters which produced the graphical illustrations of FIG. 5.

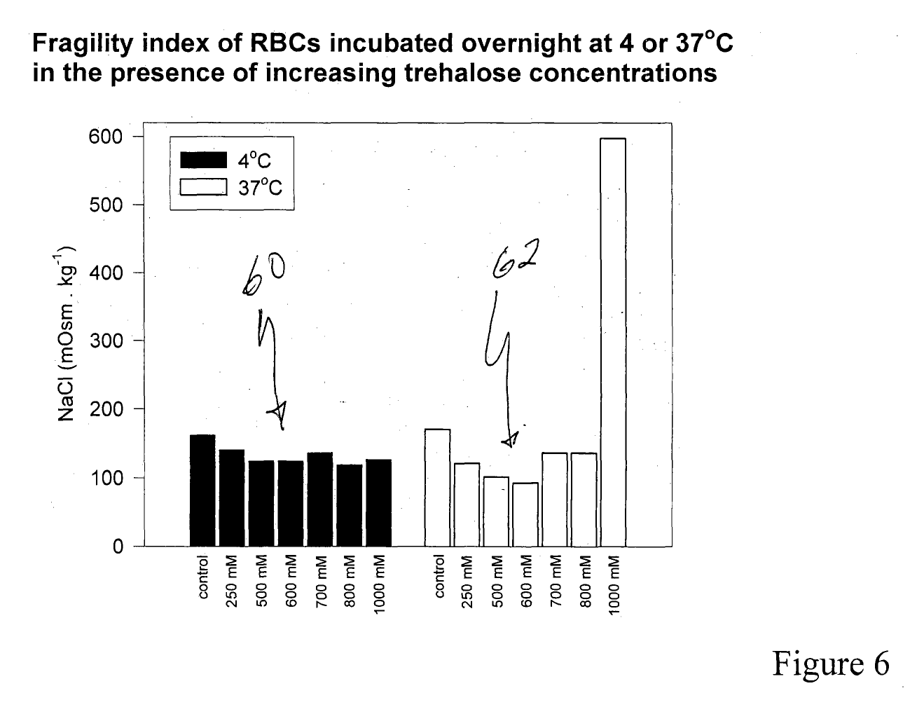

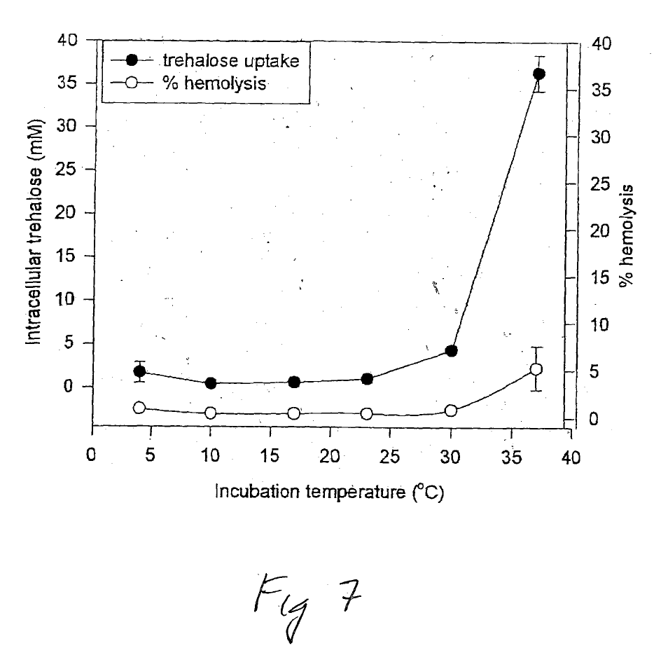

Referring now to FIG. 6, there is seen a graphical illustration of the fragility index of erythrocytic cells incubated overnight at respective temperatures of 4° C. and 37° C. in the presence of and as a function of increasing intracellular trehalose concentrations. The osmotic fragility index was generated by the extent of hemolysis as a function of the NaCl concentration. The graphical illustration of FIG. 6 represents a test for investigating the effects of hyperosmotic treatment rendering erythrocytic cells more sensitive to change in intracellular osmolarity. NaCl was loaded into erythrocytic cells from a 100 mOsm PBS buffer at loading 100 mOsm PBS buffer temperatures of 4° C. and 37° C. for extracellular trehalose concentrations of 0 mM (control cells), 250 mM, 500 mM, 600 mM, 700 mM, 800 mM and 1000 mM. Data blocks, respectively generally indicated as 60 and 62, represent the intracellular trehalose concentrations for 100 mOsm PBS solution loading temperatures of 4° C. and 37° C. The mOsm/kg values of NaCl represent extracellular NaCl osmolarity of the erythrocytic cells resulting from the transfer of NaCl from the PBS loading buffer into the erythrocytic cells. The erythrocytic cells that had been loaded in trehalose solutions (between 250 mM and 1000 mM) in 100 mOsm PBS were suspended in increasing concentrations of NaCl (between 50 and 600 mOsm NaCl). The percent hemolysis measured after resuspending the loaded cells in NaCl represents the fragility index. The data show that the erythrocytic cells were stable osmotically in trehalose media with concentrations between 250 mM and 800 mM trehalose at both 37° C. and 4° C. In 1000 mM trehalose at 37° C., there is a high increase in the fragility index suggesting that the cells were unstable in this medium (1000 mM trehalose in 100 mOsm PBS). Clearly, at moderate intracellular concentrations of trehalose, osmotic fragility as measured by a standard assay was not severely altered. Thus, erythrocytic cells may be loaded with trehalose concentrations up to about 900 mM (i.e., a trehalose concentration between 800 mM and 1000 mM). Example 3 below provides specific testing conditions and parameters which produced the graphical illustrations of FIG. 6.

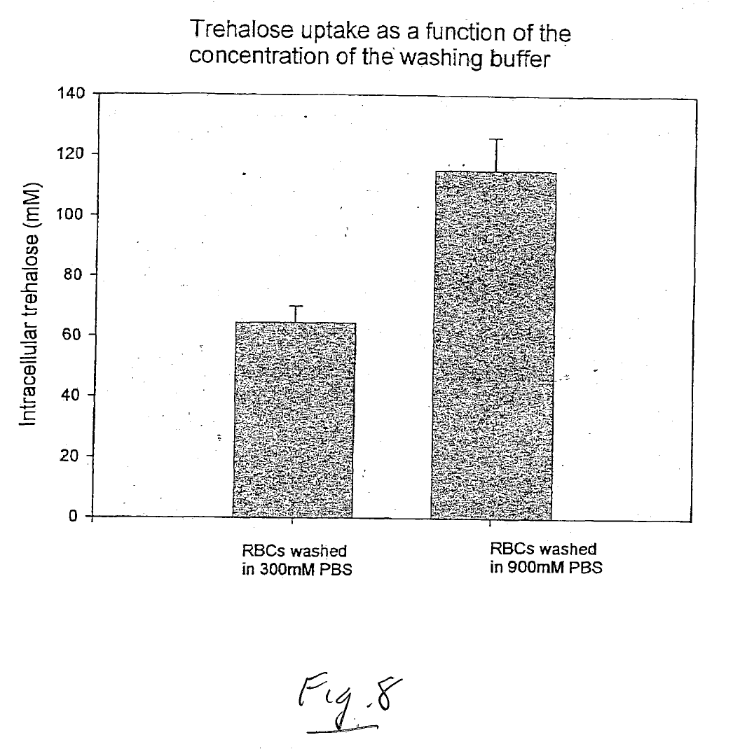

Thus, from the findings graphically illustrated in FIGS. 5 and 6, and as more fully explained in Examples 2 and 3 below, temperature of a solute loading solution has an effect in loading a solute from a solute solution into a cell. The effects of temperature, as well as cellular hemolysis, of a trehalose loading solution in loading of trehalose into a cell was tested. The test results are illustrated in FIG. 7, which is a graphical illustration of trehalose uptake (i.e., intracellular trehalose mM) and hemolysis (i.e., % hemolysis) as a function of incubation temperature (° C.). The incubation time was about 6 hours and the medium contained about 800 mM trehalose/100 mM PBS. FIG. 7 illustrates that effective loading occurs above 30° C., and that as the loading temperature of the trehalose loading solution increases, there is slight hemolysis. Example 4 below provides the more specific testing conditions and parameters which produced the graphical illustrations of FIG. 7.

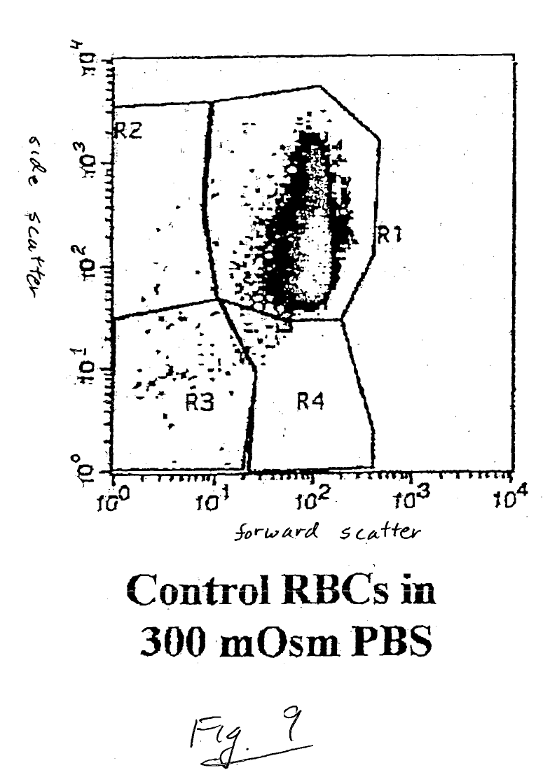

As previously indicated, after a cell (e.g., an erythrocytic cell) has been loaded with a solute (e.g., trehalose), further embodiments of the present invention provide for retaining the solute in the cells. One means for retaining solute within solute-loaded cells is to wash the cells, more specifically by washing the cells and retaining the solute in the cells during the washing. As also previously indicated, the washing of the cells is preferably with a washing buffer. It has been discovered that retention of the solute in the cells increases from about 25% to about 175% when a buffer concentration (e.g., the osmolarity of all osmotically active particles within the washing buffer solution) increases from about 50% to about 400%, more preferably from about 50% to about 150% when a buffer concentration increases from about 100% to about 300%, and most preferably from about 75% to about 125% (e.g., about 100%) when a buffer concentration increases from about 150% to about 250% (e.g., about 200%). It has been further discovered that the washing of the cells with a washing buffer includes employing a ratio of an extracellular buffer concentration (mOsm) to an intracellular trehalose concentration (mM) ranging from about 14.0 to about 4.0, more particularly from about 12.0 to about 5.0, including from about 9.0 to about 6.0 and from about 8.0 to about 7.0 (e.g., about 7.5). Thus, because solute loaded cells are hyperosmotic to a washing buffer, increasing the extracellular osmolarity increases retention of the solute, particularly during washing of the cells, as shown in FIG. 8 which graphically illustrates intracellular trehalose concentration (mM) as a function of the osmolarity of the washing buffer. As shown in FIG. 8, when the extracellular buffer concentration was increased from 300 mOsm PBS to 900 mOsm PBS during washing, the final intracellular trehalose concentration doubled. The 300 mOsm PBS had no trehalose concentration, and the 900 mOsm PBS also had no trehalose concentration. Example 5 below provides the more specific testing conditions and parameters which produced the graphical illustrations of FIG. 8.

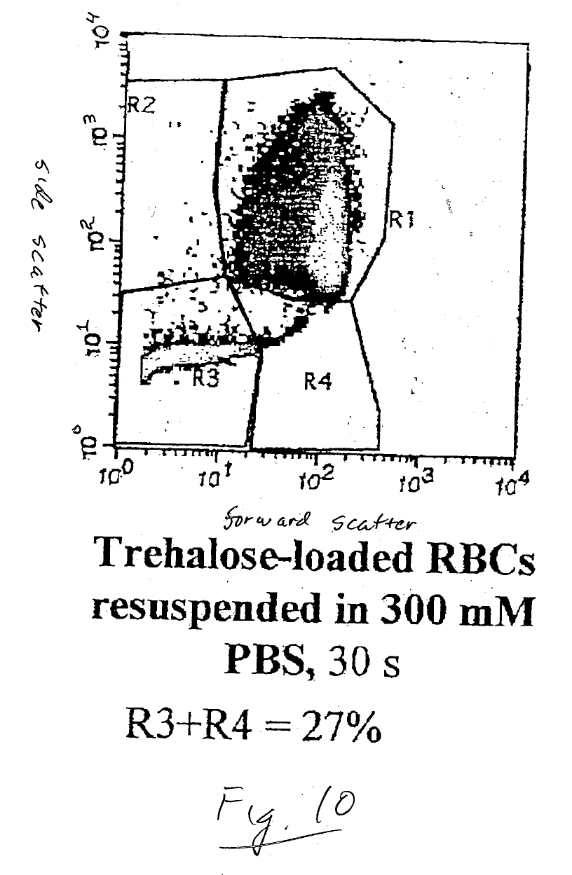

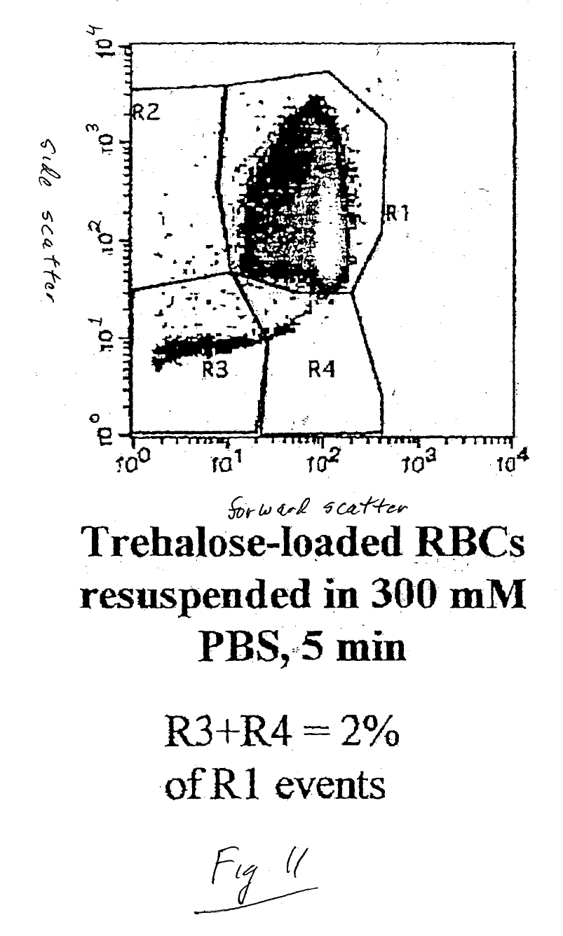

Referring now to FIGS. 9-11, there are seen the results of evaluating by flow cytometry trehalose-loaded cells and non-loaded cells (i.e., control cells) for granularity (side scatter) and cell shape (forward scatter). FIG. 9 is an evaluation by flow cytometry of non-loaded (control) human erythrocytic cells in 300 mOsm PBS (154 mM NaCl, 5.6 mM Na₂PO₄, 1.06 mM KH₂PO₄, pH 7.2) for granularity (side scatter) and cell shape (forward scatter). In the controls in gate R1, there is a discrete population of intact human erythrocytic cells with minimal signal in gates R3 and R4. Thus, the control population shows a discrete population of intact cells in R1 and a minimal number of events in R3 and R4 (representing lysed cells or fragments of cells). As best shown in FIG. 10 when trehalose loaded cells were resuspended for 30 seconds in 300 mOsm PBS having a trehalose concentration of 60 mM, about 27% of the population appears in gates R3 and R4. The larger population appearing in R3 and R4 (after the trehalose-loaded cells were resuspended for 30 seconds) indicates a group of microcytic cells that are ghost or lysed cells. Further washing with simultaneous incubation by resuspending the trehalose-loaded human erythrocytic cells for 5 minutes in 300 mOsm PBS having a trehalose concentration of 60 mM, shows a diminution of the population of microcytic cells in gates R3 and R4 to about 2%. Thus, the reduction in population of microcytic cells in gates R3 and R4 with increased incubation time (i.e., the increased from 30 seconds as shown in FIG. 10 to 5 minutes as shown in FIG. 11) reflect that the cells re-equilibrate in the washing buffer to their normal size, suggesting that the osmotically fragile and damaged cells are lysed. The small remaining fragments of damaged or lysed cells at R3 and R4 in FIG. 11 may be easily removed by centrifugation and subsequent resuspension of the cells in the washing solute or buffer. Therefore, during a short (10 min.) low speed centrifugation (500×g) cell fragments and lysed cells remain in the supernatant while intact cells were pelleted. This procedure facilitates storage of more robust cells. Example 6 below provides specific testing conditions and parameters which produced the flow cytometry pictures of FIGS. 9-11.

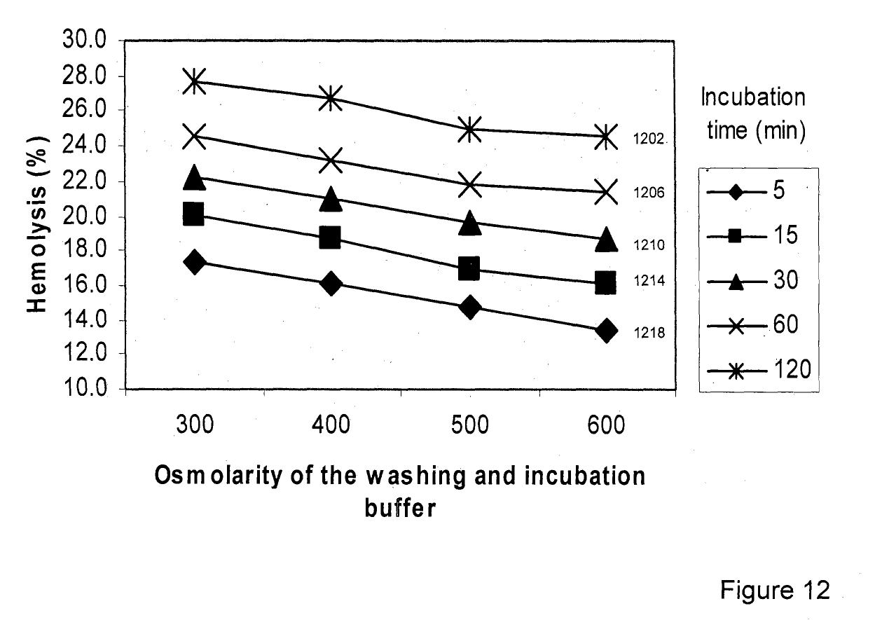

It has been discovered that the washing solute or buffer may also be used to reduce cell hemolysis following incubation. The cells are to be tested for viability immediately after incubating the cells in the designated washing buffer. The washing buffer does not have any trehalose and includes 600 mOsm PBS (308 mM NaCl, 11.2 mM Na₂HPO₄, 2.12 KH₂PO₄, and a pH of 7.2). The concentration of the NaCl and the phosphates in the PBS buffer have been increased proportionally in order to adjust the required osmolarity. Referring now to FIG. 12 there is seen a graphical illustration of hemolysis (%) vs. osmolarity of the PBS washing buffer for various incubation periods (minutes). Line 1202 represents four (4) points of the hemolysis (%) vs. osmolarity of the PBS washing and incubation buffer for an incubation time of five (5) minutes. Line 1206 represents four (4) points of the hemolysis (%) vs. osmolarity of the PBS washing and incubation buffer for an incubation time of fifteen (15) minutes. Line 1210 represents four (4) points of the hemolysis (%) vs. osmolarity of the PBS washing and incubation buffer for an incubation time of thirty (30) minutes. Line 1214 represents four (4) points of the hemolysis (%) vs. osmolarity of the PBS washing and incubation buffer for an incubation time of sixty (60) minutes. Line 1218 represents four (4) points of the hemolysis (%) vs. osmolarity of the PBS washing and incubation buffer for an incubation time of one hundred twenty (120) minutes. Lines or curves 1202, 1206, 1210, 1214, and 1218 demonstrate that as trehalose-loaded cells are washed and incubated in a solute or washing solution with increased osmolarity, there is a concomitant decrease in the percent hemolysis. At every osmolarity, hemolysis increases with time; and lower osmolarity of the PBS washing and incubation buffer results in higher hemolysis (the loss of fragile, presumably older erythrocytic cells). Example 7 below provides specific testing conditions and parameters which produced the graph of FIG. 12.

The solute or washing solution for washing the cells to reduce hemolysis has the capabilities of reducing cell hemolysis by at least about 0.50% for each 100 mOsm increase in osmolarity of the solute solution. The solute solution may reduce cell hemolysis from about 0.50% to about 8.0% for each 100 mOsm increase in osmolarity of the solute solution, more specifically from about 1.0% to about 4.0% for each 100 mOsm increase in osmolarity of the solute solution, more specifically futher from about 1.0% to about 2.0% for each 100 mOsm increase in osmolarity of the solute solution. The osmolarity of the solute or washing solution may range from about 100 mOsm to about 1500 mOsm, including from about 200 mOsm to about 1000 mOsm or from about 300 mOsm to about 600 mOsm. As indicated, a suitable solute or washing solution to reduce hemolysis may comprise a salt solution having a phosphate buffered saline 600 mOsm PBS) solution including NaCl, Na₂HPO₄, and KH₂PO₄, more specifically a PBS solution having 308 mM NaCl, 11.2 mM Na₂HPO₄, 2.12 KH₂PO₄, and a pH of 7.2.

After the cells have been effectively loaded with a solute and subsequently washed, the cells may then be contacted with a drying buffer. The drying buffer should include the solute, preferably in amounts up to about 100 mM. The solute in the drying buffer assists in spatially separating the cells as well as stabilizing the cell membranes on the exterior. The drying buffer preferably also includes a bulking agent (tb further separate the cells). Albumin may serve as a bulking agent, but other polymers may be used with the same effect. If albumin is used, it is preferably from the same species as the cells. Suitable other polymers, for example, are water-soluble polymers such as HES (hydroxy ethyl starch) and dextran.

The solute loaded cells in the drying buffer may then be dried while simultaneously cooled to a temperature below about −32° C. A cooling, that is, freezing, rate is preferably between −30° C. and −1° C./min. and more preferably between about −2° C./min to −5° C./min. Drying may be continued until about 95 weight percent of water has been removed from the cells. During the initial stages of lyophilization, the pressure is preferably at about 10×10⁻⁶ torr. As the samples dry, the temperature can be raised to be warmer than −32° C. Based upon the bulk of the sample, the temperature and the pressure it can be emperically determined what the most efficient temperature values should be in order to maximize the evaporative water loss. Freeze-dried cell compositions preferably have less than about 5 weight percent water.

After freeze drying and storage of the cells, the process of using such a dehydrated cell composition comprises rehydrating the cells. The rehydration preferably includes a prehydration step, sufficient to bring the water content of the freeze-dried cells to between about 20 weight percent and about 50 percent, preferably from about 20 weight percent to about 40 weight percent. More preferably, when reconstitution of the freeze dried cells is desired, the freeze dried cells are prehydrated in moisture saturated air at about 37° C. for about one hour to about three hours, followed by rehydration. Use of prehydration yields cells with a much more dense appearance and with no balloon cells being present. The preferred prehydration step brings the water content of the freeze-dried cells to between about 20 weight percent to about 50 weight percent. Rehydration or the prehydreated cells may be with any aqueous based solutions, depending upon the intended application.

Embodiments of the present invention will be illustrated by the following set forth examples which are being given to set forth the presently known best mode and by way of illustration only and not by way of any limitation. It is to be understood that all materials, chemical compositions and procedures referred to below, but not explained, are well documented in published literature and known to those artisans possessing skill in the art. All materials and chemical compositions whose source(s) are not stated below are readily available from commercial suppliers, who are also known to those artisans possessing skill in the art. All parameters such as concentrations, mixing proportions, temperatures, rates, compounds, etc., submitted in these examples are not to be construed to unduly limit the scope of the invention. Abbreviations used in the examples, and elsewhere, are as follows:
DMSO=dimethylsulfoxide
ADP=adenosine diphosphate
PGE1=prostaglandin E1
HES=hydroxy ethyl starch
FTIR=Fourier transform infrared spectroscopy
EGTA=ethylene glycol-bis(2-aminoethyl ether) N,N,N′,N′, tetra-acetic acid
TES=N-tris (hydroxymethyl) methyl-2-aminoethane-sulfonic acid
HEPES N-(2-hydroxylethyl) piperarine-N′-(2-ethanesulfonic acid)
PBS=phosphate buffered saline
HSA=human serum albumin
BSA=bovine serum albumin
ACD=citric acid, citrate, and dextrose
MβCD=methyl-β-cyclodextrin

### EXAMPLE 1

Washing of Platelets. Platelet concentrations were obtained from the Sacramento blood center or from volunteers in our laboratory. Platelet rich plasma was centrifuged for 8 minutes at 320×g to remove erythrocytes and leukocytes. The supernatant was pelleted and washed two times (480×g for 22 minutes, 480×g for 15 minutes) in buffer A (100 MM NaCl, 10 MM KCl, 10 mM EGTA, 10 mM imidazole, pH 6.8). Platelet counts were obtained on a Coulter counter T890 (Coulter, Inc., Miami, Fla.).

Loading of Lucifer Yellow CH into Platelets. A fluorescent dye, lucifer yellow CH (LYCH), was used as a marker for penetration of the membrane by a solute. Washed platelets in a concentration of 1-2×10⁹ platelets/ml were incubated at various temperatures in the presence of 1-20 mg/ml LYCH. Incubation temperatures and incubation times were chosen as indicated. After incubation the platelets suspensions were spun down for 20× at 14,000 RPM (table centrifuge), resuspended in buffer A, spun down for 20 s in buffer A and resuspended. Platelet counts were obtained on a Coulter counter and the samples were pelleted (centrifugation for 45 s 25 at 14,000 RPM, table centrifuge). The pellet was lysed in 0.1% Triton buffer (10 mM TES, 50 mM KCl, pH 6.8). The fluorescence of the lysate was measured on a Perkin-Elmer LSS spectrofluorimeter with excitation at 428 nm (SW 10 nm) and emission at 530 run (SW 10 nm). Uptake was calculated for each sample as nanograms of LYCH per cell using a standard curve of LYCH in lysate buffer. Standard curves of LYCH, were found to be linear up to 2000 run ml⁻¹.

Visualization of cell-associated Lucifer Yellow. LYCH loaded platelets were viewed on a fluorescence microscope (Zeiss) employing a fluorescein filter set for fluorescence microscopy. Platelets were studied either directly after incubation or after fixation with 1% paraformaldehyde in buffer. Fixed cells were settled on poly-L-lysine coated cover slides and mounted in glycerol.

Loading of Platelets with Trehalose. Washed platelets in a concentration of 1-2 10⁹ platelets/ml were incubated at various temperatures in the presence of 1-20 mg/ml trehalose. Incubation temperatures were chosen from 4° C. to 37° C. Incubation times were varied from 0.5 to 4 hours. After incubation the platelet solutions were washed in buffer A two times (by centrifugation at 14,000 RPM for 20 s in a table centrifuge). Platelet counts were obtained on a coulter counter. Platelets were pelleted (45 S at 14,000 RPM) and sugars were extracted from the pellet using 80% methanol. The samples were heated for 30 minutes at 80° C. The methanol was 10 evaporated with nitrogen, and the samples were kept dry and redissolved in H₂O prior to analysis. The amount of trehalose in the platelets was quantified using the anthrone reaction (Umbreit et al., Mamometric and Biochemical Techniques, 5th Edition, 1972). Samples were redissolved in 3 ml H₂O and 6 ml anthrone reagents (2 g anthrone dissolved in 10M sulfuric acid). After vortex mixing, the samples were placed in a boiling water bath for 3 minutes. Then the samples were cooled on ice and the absorbance was measured at 620 nm on a Perkin Elmer spectrophotometer. The amount of platelet associated trehalose was determined using a standard curve of trehalose. Standard curves of trehalose were found to be linear from 6 to 300 μg trehalose per test tube.

Quantification of Trehalose and LYCH Concentration. Uptake was calculated for each sample as micrograms of trehalose or LYCH per platelet. The internal trehalose concentration was calculated assuming a platelet radius of 1.2 μm and by assuming that 50% of the platelet volume is taken up by the cytosol (rest is membranes). The loading efficiency was determined from the cytosolic trehalose or LYCH concentration and the concentration in the loading buffer.

FIG. 1 shows the effect of temperature on the loading efficiency of trehalose into human platelets after a 4 hour incubation period with 50 mM external trehalose. The effect of the temperature on the trehalose uptake showed a similar trend as the LYCH uptake. The trehalose uptake is relatively low at temperatures of 22° C. and below (below 5%), but at 37° C. the loading efficiency of trehalose is 35% after 4 hours.

When the time course of trehalose uptake is studied at 37° C., a biphasic curve can be seen (FIG. 2). The trehalose uptake is initially slow (2.8×10⁻¹¹ mol/m²s from 0 to 2 hours), but after 2 hours a rapid linear uptake of 3.3×10⁻¹⁰ mol/m²s can be observed. The loading efficiency increases up to 61% after an incubation period of 4 hours. This high loading efficiency is a strong indication that the trehalose is homogeneously distributed in the platelets rather than located in pinocytosed vesicles.

The uptake of trehalose as a function of the external trehalose concentration is shown in FIG. 3, which graphically illustrates the internal trehalose concentration of human platelets versus external trehalose concentration as a function of temperature at a constant incubation or loading time. The uptake of trehalose is linear in the range from 0 to 30 mM external trehalose. The highest internal trehalose concentration is obtained with 50 mM external trehalose. At higher concentrations than 50 mM the internal trehalose concentration decreases again. Even when the loading buffer at these high trehalose concentrations is corrected for isotonicity by adjusting the salt concentration, the loading efficiency remains low. Platelets become swollen after 4 hours incubation in 75 mM trehalose. FIG. 4 graphically illustrates the loading efficiency of trehalose into human platelets as a function of external trehalose concentration.

The stability of the platelets during a 4 hours incubation period was studied using microscopy and flow cytometric analysis. No morphological changes were observed after 4 hours incubation of platelets at 37° C. in the presence of 25 mM external trehalose. Flow cytometric analysis of the platelets showed that the platelet population is very stable during 4 hours incubation. No signs of microvesicle formation could be observed after 4 hours incubation, as can be judged by the stable relative proportion of microvesicle gated cells (less than 3%). The formation of microvesicles is usually considered as the first sign of platelet activation (Owners et al., Trans. Med. Rev., 8, 27-44, 1994). Characteristic antigens of platelet activation include: glycoprotein 53 (gp53., a lysosomal membrane marker), PECAM-1 (platelet endothelial cell adhesion molecule-1, an alpha granule constituent), and P-selection (an alpha granule membrane protein).

### EXAMPLE 2

FIG. 5 graphically illustrates the loading efficiency of trehalose into human erythrocytic, cells as a function of external trehalose concentration at respective temperatures of 4° C. and 37° C. Erythrocytic cells were exposed to trehalose for 18 hours at either 4° C. or 37° C. The trehalose concentration in the incubation medium varied between 230 mM and 1000 mM. Each incubation buffer contained trehalose (between 230 mM and 1000 mM) and 100 mOsm PBS pH 7.2. Increase in the trehalose concentration in the loading medium results in an increase in the sugar uptake, raching abourt 100 mM cytoplasmic trehalose in erythrocytes incubated in 1000 mM trehalose and 100 mOsm PBS. At 4° C., the uptake was very limited, being about 25 mm. The trehalose intake was measured using anthrone assay and confirmed by high performance liquid chromatography. It is clear that there was substantial loading at 37° C., but not at 4° C. Furthermore, trehalose loading was not significant unless the extracellular cellular trehalose concentration gaves a hyperosmotic pressure. Since intracellular osmolarity for erythrocytic cells is about 300 mOsm, it is clear that raising the extracellular osmolarity was required for more effective loading of trehalose.

### EXAMPLE 3

FIG. 6 graphically illustrates the fragility index of erythrocytic cells incubated overnight at respective temperatures of 4° C. and 37° C. in the presence of and as a function of increasing intracellular trehalose concentrations. The osmotic fragility index was generated by the extent of hemolysis as a function of the NaCl concentration. The erythrocytic cells that had been loaded in trehalose solutions (between 250 mM and 1000 mM) in 100 mOsm PBS were suspended in increasing concentrations of NaCl (between 50 and 600 mOsm NaCl). The percent hemolysis measured after resuspending the loaded cells in NaCl represents the fragility index. The data show that the erythrocytic cells were stable osmotically in trehalose media with concentrations between 250 mM and 800 mM trehalose at both 37° C. and 4° C. In 1000 mM trehalose at 37° C., there is a high increase in the fragility index suggesting that the cells were unstable in this medium (1000 mM trehalose in 100 mOsm PBS).

### EXAMPLE 4

FIG. 7 graphically illustrates trehalose uptake (i.e., intracellular trehalose mM) and hemolysis (i.e., % hemolysis) as a function of incubation temperature (° C.). The incubation temperature was varied between 4° C. and 37° C. The erythrocytic cells were incubated for 6 hours in 800 mM trehalose in 100 mOsm PBS pH 7.2. Between 4° C. and 30° C., the cytoplasmic trehalose was very low (between 1 and 4 mM). It was considerably increased (up to 35 mM cytoplasmic trehalose) during 6 hours incubation at 37° C.

### EXAMPLE 5

FIG. 8 graphically illustrates intracellular trehalose concentration (mM) as a function of the osmolarity of the washing buffer. Earlier morphological data showed that along with discoid erythrocytic cells, there is about 20% of cells with modified shape (spherocytes and schistocytes). The issue was what was the loading capacity of these cells and how much they contribute to the amount of trehalose that was to be detected. This issue was investigated by washing the trehalose loaded erythrocytic cells (loaded at 35° C. for 16 hours in 800 mM trehalose in 100 mOsm PBS pH 7.2) in buffers with different osmolarity (300 mOsm PBS or 900 mOsm PBS) and estimating the cytoplasmic sugar concentration. The loaded cells were washed with either 300 mOsm PBS pH 7.2 (which is the isotonic medium for erythrocytic cells) or 900 mOsm PBS pH 7.2 (which matches the tonicity of the loading medium). The data in FIG. 8 illustrated that there is a decrease in the intracellular sugar concentration suggesting that a fraction of the cells was lost during the washing procedure.

### EXAMPLE 6

FIG. 9 is a forward scatter vs. a side scatter flow cytometry for non-loaded (control) human erythrocytic cells in 300 mOsm PBS. The figure shows a homogeneous population of cells in region 1 (R1) and a very small number of events in regions 3 and 4, corresponding to cells with different complexity.

FIG. 10 is a forward scatter vs. a side scatter flow cytometry for trehalose-loaded human erythrocytic cells resuspended for 30 seconds in 300 mOsm PBS having a trehalose concentration of 60 mM. The cells were loaded in 800 mM trehalose and 100 mOsm PBS at 37° C. for 16 hours. After the loading, the erythrocytes were suspended in 300 mOsm PBS. Thirty (30) seconds after resuspending in 300 mOsm PBS, in R3 and R4 there is higher population of cells with different complexity as compared to the control cells (FIG. 9). Such cells account for about 27% of the cells in R1.

FIG. 11 is a forward scatter vs. a side scatter flow cytometry for trehalose-loaded human erythrocytic cells resuspended for 5 minutes in 300 mOsm PBS having a trehalose concentration of 60 mM. The cells were loaded in 800 mM trehalose and 100 mOsm PBS at 37° C. for 16 hours. After the loading, the erythrocytes were suspended in 300 mOsm PBS. Five (5) minutes after resuspending in 300 mOsm PBS, the number of cells with different complexity was considerably decreased and accounted for only 2% of the number of events in R1. These results show that washing trehalose loaded erythrocytes with 300 mOsm PBS results in removing the cells with different complexity which possibly or probably correspond to osmotically fragile cells.

### EXAMPLE 7

FIG. 12 is a graphical illustration of hemolysis (%) vs. osmolarity of the PBS washing buffer for various washing incubation periods (min.) Line 1202 represents four (4) points of the hemolysis (%) vs. osmolarity of the PBS washing and incubation buffer for an incubation time of five (5) minutes. Line 1206 represents four (4) points of the hemolysis (%) vs. osmolarity of the PBS washing and incubation buffer for an incubation time of fifteen (15) minutes. Line 1210 represents four (4) points of the hemolysis (%) vs. osmolarity of the PBS washing and incubation buffer for an incubation time of thirty (30) minutes. Line 1214 represents four (4) points of the hemolysis (%) vs. osmolarity of the PBS washing and incubation buffer for an incubation time of sixty (60) minutes. Line 1218 represents four (4) points of the hemolysis (%) vs. osmolarity of the PBS washing and incubation buffer for an incubation time of one hundred twenty (120) minutes. Lines or curves 1202, 1206, 1210, 1214, and 1218 demonstrate that as trehalose-loaded cells are washed and incubated in a solute or washing solution with increased osmolarity, there is a concomitant decrease in the percent hemolysis. At every osmolarity, hemolysis increases with time; and lower osmolarity of the PBS washing and incubation buffer results in higher hemolysis (the loss of fragile, presumably older erythrocytic cells).

### CONCLUSION

Embodiments of the present invention provide that trehalose, a sugar found at high concentrations in organisms that normally survive dehydration, may be used to preserve biological structures in the dry state. Cells may be loaded with trehalose under the previously specified conditions, and the loaded cells can be freeze dried with excellent recovery.

While the present invention has been described herein with reference to particular embodiments thereof, a latitude of modification, various changes and substitutions are intended in the foregoing disclosure, and it will be appreciated that in some instances some features of the invention will be employed without a corresponding use of other features without departing from the scope and spirit of the invention as set forth. Therefore, many modifications may be made to adapt a particular situation or material to the teachings of the invention without departing from the essential scope and spirit of the present invention. It is intended that the invention not be limited to the particular embodiment disclosed as the best mode contemplated for carrying out this invention, but that the invention will include all embodiments and equivalents falling within the scope of the appended claims.

1. A method for reducing hemolysis in cells comprising
washing cells in a solute solution having the capabilities of reducing cell hemolysis by at least about 0.50% for each 100 mOsm increase in osmolarity of the solute solution.

2. The method of claim 1 wherein said solute solution reduces cell hemolysis from about 0.50% to about 8.0% for each 100 mOsm increase in osmolarity of the solute solution.

3. The method of claim 1 wherein said solute solution reduces cell hemolysis from about 1.0% to about 4.0% for each 100 mOsm increase in osmolarity of the solute solution.

4. The method of claim 1 wherein said solute solution reduces cell hemolysis from about 1.0% to about 2.0% for each 100 mOsm increase in osmolarity of the solute solution.

5. The method of claim 1 wherein said solute solution comprises an osmolarity ranging from about 100 mOsm to about 1500 mOsm.

6. The method of claim 1 wherein said solute solution comprises an osmolarity ranging from about 200 mOsm to about 1000 mOsm.

7. The method of claim 1 wherein said solute solution comprises an osmolarity ranging from about 300 mOsm to about 600 mOsm.

8. The method of claim 4 wherein said solute solution comprises an osmolarity ranging from about 300 mOsm to about 600 mOsm.

9. The method of claim 1 wherein said solute solution comprising a salt solution having a phosphate buffered saline (PBS) solution including NaCl, Na₂HPO₄, and KH₂PO₄.

10. The method of claim 1 wherein said solute solution comprises a PBS buffer having 154 mM NaCl, 5.6 mM Na₂HPO₄, 1.06 mM KH₂PO₄, and a pH 7.2.

11. The method of claim 1 additionally comprising removing damaged cells from the washed cells.

12. The method of claim 11 wherein removing damaged cells comprises centrifuging the washed cells.

13. The method of claim 11 additionally comprising suspending the cells in the solute solution.

14. The method of claim 1 additionally comprising loading a solute into the cells prior to washing the cells.

15. The method of claim 14 wherein said loading of the cells comprises disposing the cells in a solution having a solute concentration of sufficient magnitude to produce hyperosmotic pressure on the cells for transferring a solute from the solution into the cells.

16. The method of claim 15 wherein said solute concentration includes an extracellular cellular solute concentration for elevating extracellular osmolarity within the solution to a value which is greater than a value of the intracellular osmolarity of the cells.

17. The method of claim 15 wherein said transferring a solute is by fluid phase endocytosis.

18. The method of claim 15 wherein said solute comprises trehalose and said cells comprise erythrocytic cells.

19. The method of claim 18 wherein said transferring of trehalose from the solution into the erythrocytic-cells is without degradation of the trehalose.

20. The method of claim 18 wherein a gradient of trehalose concentration (M) within the erythrocytic cells to extracellular trehalose concentration (M) within the solution ranges from about 0.130 to about 0.200.

21. The method of claim 18 wherein a gradient of trehalose concentration (M) within the erythrocytic cell to extracellular trehalose concentration (M) within the solution ranges from about 0.04 to about 0.12.

22. The method of claim 18 wherein said solute solution has a trehalose concentration ranging from about 320 mM to about 4000 mM.

23. A cell produced in accordance with the method of claim 1.

24. The method of claim 18 wherein loading trehalose into erythrocytic cells comprises disposing the erythrocytic cells in a trehalose solution having a trehalose concentration of at least about 25% greater than the intracellular osmolarity of the erythrocytic cells for loading the trehalose into the erythrocytic cells.

25. The method of claim 14 additionally comprising
preventing a decrease in a loading efficiency gradient in the loading of the solute into the cells.

26. The method of claim 25 wherein said solute comprises an oligosaccharide and said preventing a decrease in a loading efficiency gradient in the loading of the oligosaccharide into the cells comprises maintaining a concentration of the oligosaccharide in the oligosaccharide solution below a concentration ranging from about 35 mM to about 65 mM.

27. The method of claim 25 wherein said solute comprises an oligosaccharide and said preventing a decrease in a loading efficiency gradient in the loading of the oligosaccharide into the cells comprises maintaining a positive gradient of loading efficiency to concentration of the oligosaccharide in the oligosaccharide solution.

28. The method of claim 1 additionally comprising retaining the solute in the cells during the washing.

29. The method of claim 28 wherein said washing is with a washing buffer, and retention of the solute in the cells increases from about 25% to about 175% when a buffer concentration increases from about 50% to about 400%.

30. The method of claim 28 additionally comprising washing the cells with a washing buffer wherein a ratio of an extracellular buffer concentration (mOsm) to an intracellular solute concentration (mM) ranges from about 14.0 to about 4.0.

31. A method for removing fragile cells from cells comprising:
washing cells in a solute solution having the capabilities of reducing cell hemolysis to produce washed cells including fragile cells; and
removing the fragile cells from the washed cells.

32. The method of claim 31 wherein said solute solution has the capabilities of reducing hemolysis by at least about 0.50% for each 100 mOsm increase in osmolarity of the solute solution.
